(19) United States
(12) Patent Application Publication
(10) Pub. No.: US 2007/0238092 A1
(43) Pub. Date: Oct. 11, 2007

(54) Method and Device for the Detection, Measurement and Analysis of Biological, Bioactive, Bioenergetic and Bioharmonic Signals

(76) Inventor: Pier RUBESA, Cresuz (CH)

Correspondence Address: BLANK ROME LLP, 600 NEW HAMPSHIRE AVENUE, N.W., WASHINGTON, DC, 20037, US

(21) Appl. No.: 11/739,476

(22) Filed: Apr. 24, 2007

Related U.S. Application Data
(63) Continuation of application No. PCT/EP05/55780, filed on Nov. 7, 2005.

(30) Foreign Application Priority Data
Nov. 5, 2004 (EP) 2004EP-105569

(51) Int. Cl. C12Q 1/00; C12M 3/00

(52) U.S. Cl. 435/4; 435/287.1

(57) ABSTRACT

Device and method for the detection of a bioharmonic field emanating from an organism. Includes a base oscillator, for radiating an audio frequency to the organism under test, and a modulation oscillator, for combining an electromagnetic signal received from the organism under test with an audio carrier.

### REFERENCE DATA

This application is a continuation of International Patent Application 2005WO-EP055780 (WO06048456) filed on Nov. 7, 2005, claiming priority of European patent application 2004EP-105569 filed on Nov. 5, 2004, the contents whereof are hereby incorporated by reference.

### FIELD OF THE INVENTION

This invention relates to a method and device for the detection, measurement and analysis of biological, bioactive, bioenergetic and bioharmonic signals.

### DESCRIPTION OF RELATED ART

Today, it is readily accepted in many scientific circles that biological matter produces a characteristic vibration pattern, called a bioactive field, or at times referred to as a bioharmonic field, with reference to its harmonic nature. These fields consist of low-frequency electromagnetic, electrostatic and mechanical vibrations that are characteristic of and related to the nature, activity and behaviour of the biological matter. These fields surround all biological matter and interact with the surrounding environment and other biological forms. They extend in time as well as in space and encompass all the biological activity for the entire lifetime of a biologic form, where they can be called a “life envelope”.

The term bioactive, bioenergetic or bioharmonic indicates here a detectable signal produced by a biological entity, like for example live organisms, or by biologically supportive environments, for example water or soil or, in general, any environment able to sustain life. A bioharmonic signal relates to a vibration-induced signal comprising a series of overtones whose reciprocals are in arithmetic progression.

Within the biological realm, the frequency of the waves of the biological signals, bioactive fields and bioenergetic or bioharmonic fields can be placed in the infrasonic, audio, and ultrasonic bands of the spectrum. It is recognized that biological organisms produce such low frequency vibrations as the result of numerous macroscopic and microscopic functions and processes.

In the course of a lifetime the biological organism will experience an interaction with a wide range of electromagnetic waves. Some of these are related to the life processes of the organism itself (i.e. birth, growth, reproduction, illness); some others may be the result of environmental factors (i.e. other organisms, earth current, cosmic waves); or of manmade activity (i.e. radio broadcasts, electrical equipment, microwave). The interplay of these various waves leaves detectable effects on the physical and biological characteristics of a biological organism, as well as on its bioactive, bioenergetic or bioharmonic field.

A bioactive, bioenergetic or bioharmonic field is exhibited not only by living beings, but also by organically supportive environments, particularly by those materials able to support life such as water, soil, minerals, and organic molecules. Water, in particular is able to retain impression of the biologically active substances it has been in contact with. In the following the term “organism” will be employed for designating both living beings in the conventional sense and those materials able to support life and which react with the biological signals, and bioactive, bioenergetic or bioharmonic fields.

### BRIEF SUMMARY OF THE INVENTION

It is a goal of the present invention to provide a device and a method of detecting and analyzing such bioactive, bioenergetic or bioharmonic fields. Such goal is attained by the device and method of the independent claims in the corresponding categories.

### BRIEF DESCRIPTION OF THE DRAWINGS

FIG. 1 represents a schematic diagram of a device according to the invention.

FIG. 2 represents a schematic diagram of the device of FIG. 1 comprising a microprocessor-controlled digital acquisition system.

FIG. 3 depicts diagrammatically a possible realization of a connection network according to the invention.

FIG. 4 represents an example of a 3-D spectrum obtained by the device of the invention.

FIGS. 5 and 6 represent positive and negative components of said 3-D spectra.

FIGS. 7 and 8 show the spectra obtained from a healthy and a sick plant sample, respectively.

FIG. 9 represents a further embodiment of the present invention.

### DETAILED DESCRIPTION OF THE INVENTION

The detection device 16 of the invention, depicted diagrammatically on FIGS. 1-3, comprises a base signal generator 30 capable of generating a periodic electric oscillation having the desired characteristics. The base signal generator should produce periodic waveforms in the frequency range where the bioharmonic signals are expected, typically comprised between 0.01 Hz to 300 kHz, although the invention is not limited to signals within these limits. The wave shape can be a sine wave or a square wave, or an arbitrary periodic waveform having any desired spectral content. Preferably the characteristics of the signal generated by the base signal generator 30, for example amplitude, frequency and waveform, can be determined at will by a digital control input 42. However this desirable feature is not a limitation of the invention and the base signal generator may include instead manual setting means for changing signal characteristics, or some or all of the characteristics of the base signal may be fixed.

The signal generated by the base generator 30, present on the terminal 31, is fed to a connection network 32 which is connected both to an antenna or electrode 34 and to the input of an amplifier 36. The antenna or electrode 34 is used both as a transmitting and as anature, activity and behaviour receiving device, as it is used both to induce a low frequency electromagnetic, electrostatic or mechanical vibration signal and, at the same time, to capture the variation in the propagated field.

Different kinds of antennas or electrodes, for example wire or coil antennas or electrodes, or plate antennas or electrodes, coupling to the mechanical vibration, electric and/or to the magnetic component of the bioactive, bioenergetic or bioharmonic field, can be employed in the framework of the present invention. The nature and the dimension of the antenna or electrode can be adapted to the characteristics of the organism under study (i.e. an antenna used for testing a tree vs. an antenna used to analyze a cell culture).

According to the needs the antenna or electrode could be replaced by an appropriate probe or coupling means: for example a vibration transducer, coupling to the mechanical vibration, electric and/or to the magnetic component of the bioactive, bioenergetic or bioharmonic field.

The connection network, an example or realization of which is visible on FIG. 1, comprises preferably a resonant tuning coil 62 and a variable element, for example the variable resistor 60, for tuning the device to the specific frequency bands of interest. The circuit of FIG. 3 represents only one possible way of realizing the connection network 32 and may be replaced by a number of other networks, including networks comprising variable capacitors and inductors, which would be too long to enumerate here.

The combined signal at the output 33 of the connection network 32 is treated by an amplifier 36, for raising its level to an appropriate value, and then led to a modulation input of a signal oscillator 38. The signal oscillator 38 is a high quality variable signal generator, for example a VCO (Voltage Controlled Oscillator), to produce a predefined carrier oscillation, for example, but not necessarily, in the range from 0.01 Hz to 300 kHz, at the output 40, which is frequency-modulated by the signal of the modulation input 37.

As for the base oscillator 30, the characteristics of the carrier frequency of the signal oscillator 38 can be fixed, or settable by manual controls, or by an analogue or digital control terminal not represented, according to the circumstances.

The signal emitted by the biological sample under test is then captured by the antenna or electrode 34 and filtered by the network 32. The modifications produced on the carrier signal by the introduction of the control voltage 37 cause displacements of the timbral, spectral and time characteristics of the signal available at the output 40.

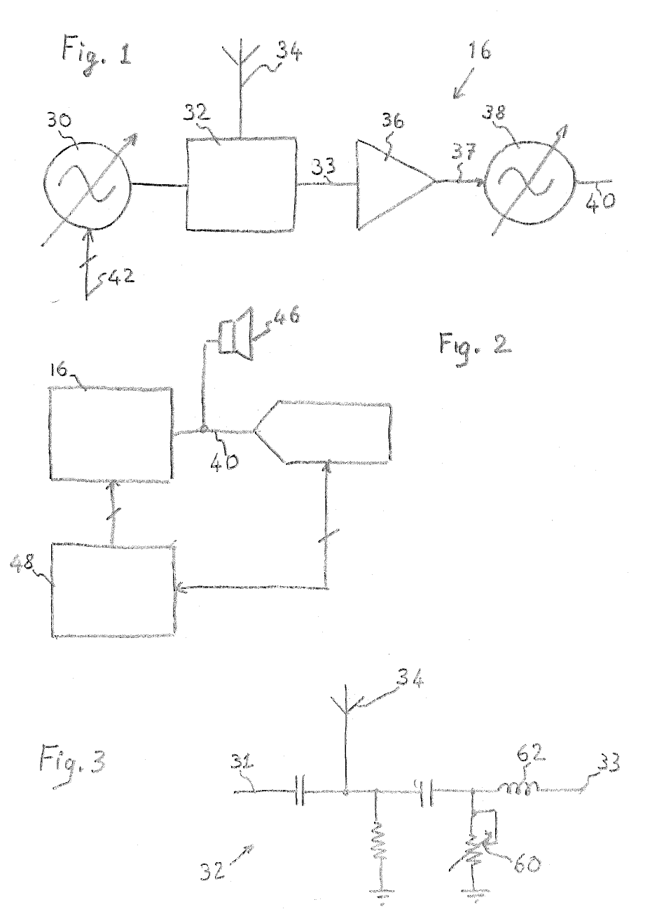

The inclusion of the bioactive, bioenergetic or bioharmonic field receiver 16 in a computerized analysis system is now discussed with reference to the FIG. 2.

The output 40 of receiver 16 is connected to the input of an ADC (Analogue to Digital Converter) and transferred to a computer system 48, for further analysis. The same computer systems 48 preferably also controls the working parameters of the receiver 16, like for example the frequency and the waveform of the base oscillator 30 and of the signal oscillator 38, and the tuning of the network 32. Additionally the output signal 40 is also fed do the loudspeaker 46 for direct aural appreciation.

The digitized signal is stored on a permanent memory of the computer system 48, and can be played back later, or analyzed by appropriate software routines running on the computer system 48, satisfactorily results are obtained in particular by the application of Chebyshev digital filtering and FFT analysis.

The bioactive, bioenergetic or bioharmonic signal wavefront comprises a number of key harmonic or enharmonic components that correspond to the prevalent vibration found in a biological signal that are specific to certain biological, behavioural or biochemical properties or processes of the substance being measured.

A convenient format for presentation and comparison of the bioactive signals is a 3-dimensional graph in which the X-axis corresponds to time, the Y-axis to frequency and the vertical Z-axis corresponds to amplitude. FIG. 4 presents an example of this presentation.

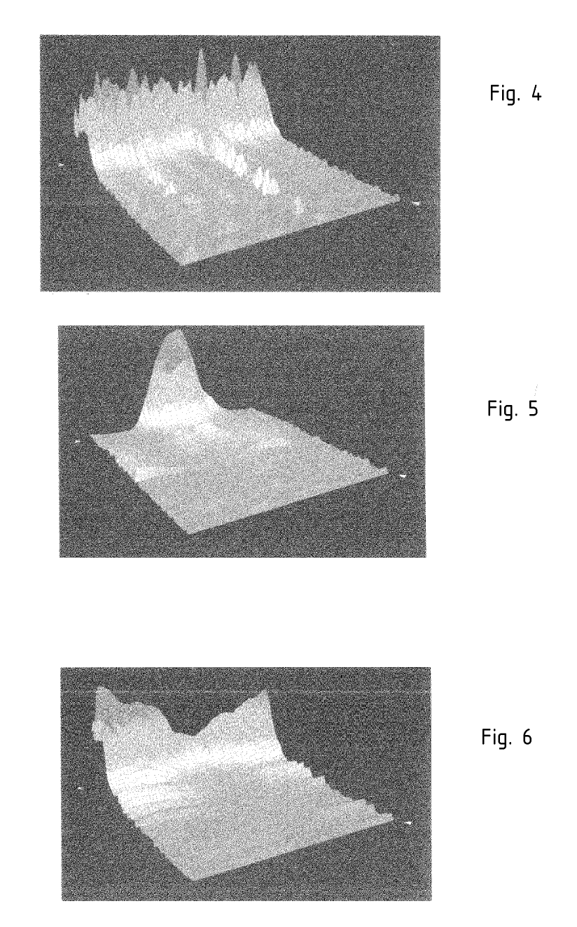

With respect to the spectral content of the base oscillator signal, the bioactive signal contains two distinct characteristics where the signal is either positive or negative. The positive part corresponds to those spectral components that are enhanced by the bioharmonic field (FIG. 5). The negative part (FIG. 6) corresponds, on the contrary, to components which are absorbed by the bioharmonic field.

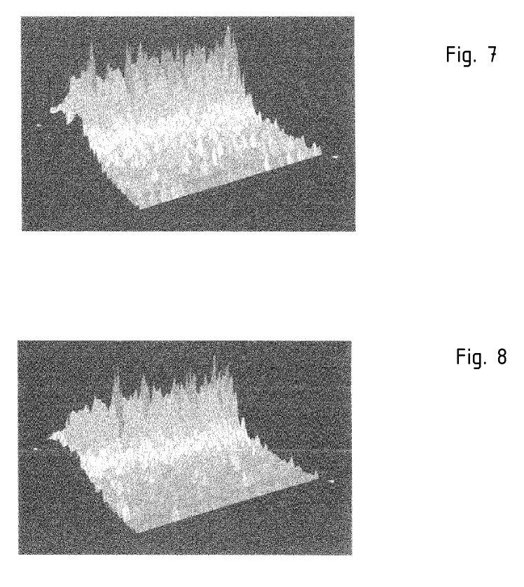

The device of the invention can usefully be employed for detecting and monitoring disease conditions in biological systems, for example in plants. The 3-dimensional graphs of FIG. 7 show the bioharmonic signal read from a healthy plant sample, whereas FIG. 8 displays the corresponding data, obtained from a sick plant sample. The device of the invention is also able to pick-up the specific signatures of viral, bacterial or fungal plant diseases. The differences in plant health state can also be clearly perceived through the loudspeaker 46.

Also the ripeness grade and the sugar content of fruit and vegetable can be assessed from the differences in the bioharmonic signals recorded by the apparatus of the invention. The invention has proved useful in detecting pathological conditions in animal cellular samples as well as in plant organisms.

The device of the invention allows also the detection of biologically derived substances, like tinctures, plant extracts, minerals, and toxic substances like pesticides, in living systems, water and soil. Thanks to this ability, the device of the invention allows distinguishing biologically-growth food farm products from and intensive-growth ones, and can detect minerals and toxins in foods, meats, cheese and beverages. The device and method of the present invention can therefore distinguish biologically grown farm food products from conventional ones.

GMO (genetically modified organisms) also are distinguishable from ordinary ones, by the differences in the respective bioharmonic fields.

Another important application of the device of the invention is the monitoring and diagnostics of water quality, for example in food, cosmetic or pharmaceutical industry or in water treatment plants.

An embodiment of the present invention having the ability to resolve spatial patterns of the bioenergetic field will now be described.

We have already seen that the single antenna or electromagnetic probe can be used both for emitting and receiving electromagnetic, electrostatic or mechanical vibration signals which are representative of the bioenergetic field emanated from a living organism or biological entity. This arrangement however provides no information as of the localization of the vibration sources, which are important in the case of measurements done on large, extended bodies (e.g. a human body).

This embodiment of the invention may also be employed in applications involving several vibration sources, for example for monitoring and diagnosing the bioactive, bioenergetic or bioharmonic condition of several trees in an orchard.

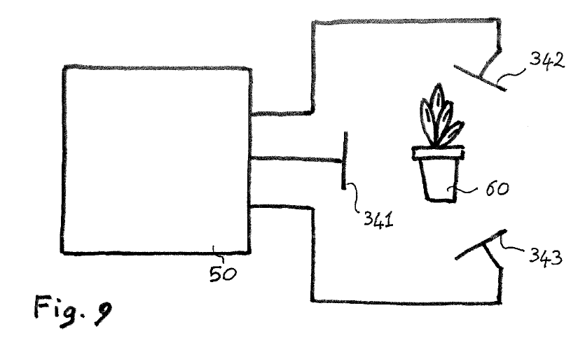

According to this embodiment, represented in FIG. 9, the detector comprises several probes, electrodes or antennas, preferably at least three probes 341, 342, 433, connected to a detection module 50. The probes 341, 343 are preferably placed around the biological system under test 60, although other spatial distributions are possible.

The detection module 50 is arranged to use each of the probes 341-343 as an emitting and receiving electrode antenna for stimulating and detecting a bioactive, bioharmonic or bioenergetic electromagnetic, electrostatic or mechanical vibration surrounding the biological system 60, much in the same way as the devices previously described having one single probe. However the module 50 is arranged for extracting localization information on the bioenergetic source 60, by weighting of the signal intensity of corresponding spectral components of the vibration signal detected by each of the individual probes 341-343. By this device, the source of vibrations, e.g. a part of a plant having developed a disease, can be localized.

Three independent probes, as represented here, represent a useful compromise, as three independent intensities allow the determination of the position of a point source in a plane. However this embodiment could use an arbitrarily large number of probes according to needs, for example if a very fine spatial discrimination is required.

On the other hand the same principle could also be applied to a system with two probes only. In this case, which may be of interest when the disposition of the sources is particularly simple, the system is able to localize only one spatial coordinate of the vibration source.

1. A device for the detection of a vibration field, for example an electromagnetic, electrostatic or mechanical vibration field, emanated from an organism or from an organically supportive environment; comprising:
a base oscillator, for generating a base signal at low frequency;
a vibration coupling means for radiating a vibration field and receiving a vibration signal emanated from said organism;
a signal oscillator for generating an output audio signal having a modulation input on which a modulation signal derived from said antenna is applied.

2. The device of claim 1, further comprising a network connected to an output of said base oscillator and to said vibration coupling means, and providing a combined output signal.

3. The device of claim 2, wherein said network comprises a variable element.

4. The device of claim 1, comprising an amplifier for amplifying said combined output signal and whose output is delivered to said modulation input.

5. The device of claim 1, further comprising an ADC and a computer system for digitizing and analyzing said output audio signal, said computer system being programmed for extracting a FFT of said output audio signal.

6. The device of claim 5, wherein said computer system is programmed for applying a digital filter to said output audio signal.

7. The device of claim 5, wherein said computer system is arranged for modifying operational parameters of said base oscillator or of said signal oscillator.

8. The device of claim 1, further comprising a loudspeaker for playing back said output audio signal.

9. The device of claim 1, characterized in that it comprises a multiplicity of vibration coupling means for radiating a vibration field and receiving a vibration signal emanated from said organism.

10. The device of claim 1, wherein the vibration coupling means consist in one or more antennas or electrodes.

11. A method of detecting and analyzing an electromagnetic, electrostatic or mechanical vibration field emanated from an organism comprising the steps of:
providing a base electrical oscillation at low frequency of predefined frequency and waveform;
transmitting said base oscillation to a space adjacent said organism by one vibration coupling means;
receiving an electromagnetic, electrostatic or mechanical vibration field, emanated from said organism, by said vibration coupling means and providing a combined output signal;
combining said combined output signal with a carrier signal of predetermined frequency and waveform, for obtaining an output audio signal;
analyzing said output audio signal.

12. The method of claim 11, wherein said output audio signal is analyzed acoustically.

13. The method of claim 11, wherein said output audio signal is digitized and analyzed by a FFT computer program.

14. The method of claim 11, further comprising a step of tuning a variable element comprised in a network providing said base oscillation to said vibration coupling means.

15. The method of claim 11, applied to the detection and monitoring of a pathological condition in an animal or vegetal organism.

16. The method of claim 11, applied to the detection and monitoring of impurities in water or in soil or in food or in beverages.

17. The method of claim 11, applied to the detection of food quality.

18. The method of claim 11, wherein several antennas or electrodes are provided for transmitting said base oscillation and for receiving said electromagnetic field,
wherein each combined signal available at each of the antennas or electrodes is combined with a carrier signal for producing several audio signals obtained by the vibration coupling means,
comprising a step of extracting a localization information by weighting the intensities of spectral components of the audio signals obtained by the vibration coupling means.
